SEQUENCE LISTING (18 sequences)

SEQ ID NO: 1 - protein, 276 aa, Homo sapiens
DSEEDEEHTIITDTELPPLKLMHSFCAFKADDGPCKAIMKRFFFNIFTRQCEEFIYGGCEGNQNRFESLEECKKMCTRDNANRIIKTTLQQEKPDFCFLEEDPGICRGYITRYFYNNQTKQCERFKYGGCLGNMNNFETLEECKNICEDGPNGFQVDNYGTQLNAVNNSLTPQSTKVPSLFEFHGPSWCLTPADRGLCRANENRFYYNSVIGKCRPFKYSGCGGNENNFTSKQECLRACKKGFIQRISKGGLIKTKRKRKKQRVKIAYEEIFVKNM

SEQ ID NO: 2 - protein, 277 aa, homo sapiens
ADSEEDEEHTIITDTELPPLKLMHSFCAFKADDGPCKAIMKRFFFNIFTRQCEEFIYGGCEGNQNRFESLEECKKMCTRDNANRIIKTTLQQEKPDFCFLEEDPGICRGYITRYFYNNQTKQCERFKYGGCLGNMNNFETLEECKNICEDGPNGFQVDNYGTQLNAVNNSLTPQSTKVPSLFEFHGPSWCLTPADRGLCRANENRFYYNSVIGKCRPFKYSGCGGNENNFTSKQECLRACKKGFIQRISKGGLIKTKRKRKKQRVKIAYEEIFVKNM

SEQ ID NO: 3 - protein, 22 aa, homo sapiens
TKRKRKKQRVKIAYEEIFVKNM

SEQ ID NO: 4 - protein, 23 aa, homo sapiens
KTKRKRKKQRVKIAYEEIFVKNM

SEQ ID NO: 5 - protein, 14 aa, homo sapiens
TKRKRKKQRVKIAY

SEQ ID NO: 6 - protein, 187 aa, homo sapiens
QQEKPDFCFLEEDPGICRGYITRYFYNNQTKQCERFKYGGCLGNMNNFETLEECKNICEDGPNGFQVDNYGTQLNAVNNSLTPQSTKVPSLFEFHGPSWCLTPADRGLCRANENRFYYNSVIGKCRPFKYSGCGGNENNFTSKQECLRACKKGFIQRISKGGLIKTKRKRKKQRVKIAYEEIFVKNM

SEQ ID NO: 7 - protein, 27 aa, homo sapiens
GFIQRISKGGLIKTKRKRKKQRVKIAY

SEQ ID NO: 8 - protein, 22 aa, homo sapiens
NFQRKEKREVIYKVKTKIKAMR

SEQ ID NO: 9 - protein, 37 aa, homo sapiens
LLGDFFRKSKEKIGKEFKRIVQRIKDFLRNLVPRTES

SEQ ID NO: 10 - protein, 35 aa, homo sapiens
GFIQRISKGGLIKTKRKRKKQRVKIAYEEIFVKNM

SEQ ID NO: 11 - protein, 11 aa, homo sapiens
TKRKRKKQRVK

SEQ ID NO: 12 - protein, 15 aa, Artificial Sequence
  N-terminal sequencing result
GTQLNAVNNSLTPQS

SEQ ID NO: 13 - protein, 15 aa, artificial sequence
  N-terminal sequencing result
NAVNNSLTPQSTKVX

SEQ ID NO: 14 - protein, 15 aa, Artificial Sequence
  N-terminal sequencing result
ADSEEDEEHTIITDT

SEQ ID NO: 15 - protein, 15 aa, Artificial Sequence
  N-terminal sequencing result
GTQLNAVNNSLXXXX

SEQ ID NO: 16 - protein, 15 aa, artificial sequence
  N-terminal sequencing result
NXVNXXLTXXXXXXX

SEQ ID NO: 17 - protein, 15 aa, Artificial Sequence
  N-terminal sequencing result
EFHGPSWXLTPADRG

SEQ ID NO: 18 - protein, 15 aa, Artificial Sequence
  N-terminal sequencing result
ADSEEDEXXXXXXXX

(19) United States
(12) Patent Application Publication
(10) Pub. No.: US 2009/0176701 A1
(43) Pub. Date: Jul. 9, 2009

(54) Anti-microbial agents that interact with the complement system

(76) Inventors: Sabine Schirm, Emeryville, CA (US); Stephen Hardy, Emeryville, CA (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/988,906

(22) PCT Filed: Jul. 24, 2006
(86) PCT No.: PCT/US2006/028804

§ 371 Date: Jan. 16, 2008

Related U.S. Application Data
(60) Provisional application No. 60/702,049, filed on Jul. 22, 2005.
(60) Provisional application No. 60/722,512, filed on Sep. 29, 2005.

(51) Int. Cl. A61K 38/16; C07K 14/00; A61P 37/04

(52) U.S. Cl. 514/12; 530/324; 514/13; 530/326

(57) ABSTRACT

Anti-microbial therapeutic agents that act via a novel method to treat infection are compounds which may comprise a peptide with natural or non-natural amino acids, or a small molecule. The agent can bind to the surface of a microorganism and productively fix complement in order to cause lysis of the microorganism via the assembly of a membrane attack complex, thereby triggering removal of the microbe by phagocytosis. The agents may be fragments of TFPI e.g. from the C-terminus region.

All patents, patent applications, online information and references cited in this disclosure are incorporated herein by reference in their entireties.

### BACKGROUND OF THE INVENTION

Microbial infections can be caused by a wide range of microbes such as bacteria, fungi and viruses resulting in mild to life-threatening illnesses that require immediate intervention. Common bacterial infections include pneumonia, ear infections, diarrhea, urinary tract infections, and skin disorders. Common viral infections include influenza A and B, respiratory syncytial virus, Hepatitis C and chicken pox whilst common fungal infections include skin disorders. There is a continuing need in the art for effective methods of treating microbial infections and/or improving the current methods of treating these infections.

### DESCRIPTION OF THE INVENTION

The present application describes anti-microbial therapeutic agents that act via a novel method to treat infection. The therapeutic agent is a compound which may comprise a peptide with natural or non-natural amino acids, or a small molecule. The agent is effective against extracellular microorganisms such as bacteria, fungi or virus infected cells. The microorganism may be prokaryotic, eukaryotic or single cellular. The therapeutic agent can work by binding to the surface of the microorganism and interacting with the components of the complement system to kill the microbe. In one embodiment, the therapeutic agent interacts synergistically with the components of the complement system to kill the microbe. In another embodiment the therapeutic agent binds to the surface of the microorganism and productively fixes complement in order to opsonize the microbe. In a further embodiment, the therapeutic agent binds to the surface of the microorganism and productively fixes complement in order to cause lysis of the microorganism via the assembly of a membrane attack complex (MAC). This triggers the removal of the microbe by phagocytosis.

Thus the invention provides a compound for treating microbial infection in an animal having a complement system, wherein the compound binds to the microbial surface and interacts with components of the complement system present in the animal (such as the C1q component) to kill microbes.

The compound may opsonize and/or cause lysis of the microbe.

The compound may act synergistically with components of the complement system present in the animal, such as C1q. Thus the anti-microbial effect of the compound may be greater in the presence of the complement system component(s) than in their absence. Preferably, the anti-microbial effect of the compound is greater than the aggregate effect of the peptide alone and the complement system component(s) alone.

Particular compounds of interest are derived from tissue factor pathway inhibitor (TFPI), as described in more detail below. Further compounds may be identified by screening methods e.g. by comparing the anti-microbial effect of a compound in the absence and presence of components of complement.

### TFPI and TFPI Analogs

TFPI is a powerful anticoagulant thought to have anti-inflammatory activity [1]. TFPI can be used to inhibit angiogenesis associated with, for example, tumors [2].

The protein has several principal domains: three serine protease inhibitor domains of the Kunitz type (K1,K2 and K3), an N-terminal domain (NTD), and a C-terminal domain (CTD). The K1 domain inhibits clotting factor VIIa-tissue factor (TF) complex. The K2 domain inhibits factor Xa. Thus far no serine protease has been associated with K3, but recent experiments suggest that K3 functions in binding TFPI to a GPI anchored receptor on cell surfaces [3]. The CTD is also involved in cell association, heparin binding, and optimal Xa inhibition.

“TFPI” as used herein refers to the mature serum glycoprotein having the 276 amino acid residue sequence shown in SEQ ID NO:1 and a molecular weight of about 38,000 Daltons without glycosylation. The native protein has a molecular weight of 45,400 Daltons when glycosylation is present [4]. The cloning of the TFPI cDNA is described in reference 5. TFPI used in the invention may be non-glycosylated or glycosylated.

A “TFPI analog” is a derivative of TFPI modified with one or more amino acid additions or substitutions, for example from one to eighty (generally conservative in nature and preferably in non-Kunitz domains or in the C-terminal portion of the protein), one or more amino acid deletions, for example from one to eighty (e.g., TFPI fragments), or the addition of one or more chemical moieties to one or more amino acids, so long as the modifications do not destroy TFPI biological activity. The activity that is not destroyed can include TFPI's anticoagulant activity and/or its anti-bacterial activity, as well as its activity in the prothrombin assay.

Preferably, TFPI analogs comprise all three Kunitz domains. TFPI and TFPI analogs can be either glycosylated or non-glycosylated.

To maintain anti-bacterial activity, it is preferred that a TFPI analog should retain its CTD, as this region is where the anti-bacterial activity has been localized. Typically, it is preferred to retain substantially all of the amino acids downstream of the most-downstream thrombin cleavage site in TFPI (e.g. downstream of amino acid 254 of SEQ ID NO: 1, in which thrombin cleaves between residues 254 & 255). At least 50% (e.g. ≧60%, ≧70%, ≧80%, ≧90%, ≧92%, ≧94%, ≧96%, ≧98%, ≧99%, or more) by number of the TFPI analog molecules in a composition should be uncleaved at the thrombin cleavage site present between amino acids 254 and 255 of TFPI.

A preferred TFPI analog is N-L-alanyl-TFPI (ala-TFPI), whose amino acid sequence is shown in SEQ ID NO:2. Ala-TFPI is also known under the international drug name “tifacogin”. The amino terminal alanine residue of ala-TFPI was engineered into the TFPI sequence to improve E. coli expression [6]. Endogenous TFPI is secreted and expressed with a signal peptide. The amino terminal methionine is part of the signal peptide and not part of the mature TFPI. Other analogs of TFPI are described in reference 7. TFPI analogs possess some measure of the activity of TFPI as determined by a bioactivity assay (for example, see refs. 8 & 9 as described below).

TFPI has three thrombin cleavage sites: (i) between Lys-86 & Thr-87, between K1 & K2; (ii) between Arg-107 & Gly-108 (the reactive site toward factor Xa in K2); and (iii) between Lys-254 & Thr-255 in the C-terminal basic region. The inventors have found that anti-bacterial activity of TFPI resides in the CTD, and in particular in the region proximal to and/or downstream of the thrombin cleavage site between Lys-254 and Thr-255 in SEQ ID NO:1. As the cleaved TFPI, lacking its CTD, has little activity in blood assays then the invention provides a TFPI analog in which this thrombin cleavage site has been removed e.g. by site-directed mutagenesis. The CTD of these analogs cannot be cleaved by thrombin, giving a molecule that can retain its anti-bacterial activity for longer periods than natural TFPI.

Thus the invention provides: (1) a TFPI analog, wherein the analog lacks the thrombin cleavage site found near the C-terminus of natural TFPI; (2) a TFPI analog, wherein the analog lacks the thrombin cleavage site present between amino acids Lys-254 and Thr-255 of natural TFPI; (3) a TFPI analog, wherein the analog comprises (i) at least one Kunitz domain and (ii) a C-terminal region, but wherein the analog does not have a thrombin cleavage site between its most C-terminal Kunitz domain and the C-terminal region; (4) a TFPI analog, wherein the analog cannot be cleaved by thrombin to give a N-terminal polypeptide that includes a Kunitz domain and a C-terminal polypeptide that does not include a Kunitz domain; (5) a TFPI analog, wherein the analog contains fewer than two (i.e. one or none) Lys-Thr dipeptides.

The natural cleavage site (Lys-Thr) can be removed in various ways. For instance, the lysine and/or the threonine can be substituted with different amino acids to give a dipeptide that is not recognized by thrombin. As an alternative, the lysine and/or the threonine can be deleted. As a further alternative, one or more amino acids can be inserted between the lysine and the threonine. After the modification has been made, the TFPI analog can be incubated with thrombin in a test digestion to confirm that the natural C-terminus cleavage no longer takes place.

The invention also provides: (1) a TFPI analog, wherein the analog includes Kunitz domain 3, but lacks the C-terminus domain; (2) a TFPI analog, wherein the analog is a TFPI that has been truncated by up to q (q is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40) amino acids from the C-terminus. C-terminus truncation of TFPI has been reported previously, but this has usually been in combination with deletion of K3.

Polypeptides

TFPI has three thrombin cleavage sites: (i) between Lys-86 & Thr-87; (ii) between Arg-107 & Gly-108; and (iii) between Lys-254 & Thr-255. The inventors have found that the anti-bacterial activity of TFPI resides in the C-terminal basic region and in particular in the region proximal to and/or downstream of the thrombin cleavage site between Lys-254 and Thr-255 in SEQ ID NO:1. Cleavage at this site liberates a 22 amino acid peptide (SEQ ID NO:3) which has been shown to have anti-bacterial activity and may bind to bacterial LPS. Thus the invention provides peptides based on the CTD of TFPI, for use as anti-bacterial agents, for use in methods of treatment of bacterial infections, and for use in manufacture of medicaments for treating such infections. The invention further provides peptides based on the CTD of TFPI, for use as anti-microbial agents, for use in methods of treatment of microbial infections and for use in manufacture of medicaments for treating such infections. These peptides are particularly active in the presence of blood.

Thus the invention provides: (1) a polypeptide consisting of amino acid sequence SEQ ID NO:3 (peptide #1); (2) a polypeptide comprising amino acid sequence SEQ ID NO:3, provided that the polypeptide is not TFPI or a TFPI analog; (3) a polypeptide comprising amino acid sequence SEQ ID NO:3, provided that the amino acid (if one is present) to the N-terminus of SEQ ID NO:3 is not Lys; (4) a polypeptide comprising an amino acid sequence that is at least 50% (e.g. ≧60%, ≧70%, ≧80%, ≧85%, ≧90%, ≧92%, ≧94%, ≧96%, ≧98%, or more) identical to SEQ ID NO: 3; (5) a polypeptide comprising amino acid sequence SEQ ID NO:3, provided that at least one of the amino acids in said SEQ ID NO:3 is a D-amino acid; (6) a polypeptide comprising a fragment of at least 3 (e.g. 4, 5, 6, 7, 8, 9, 10, 11, 12, 13, 14, 15, 16, 17, 18, 19, 20 or 21) consecutive amino acids of amino acid sequence SEQ ID NO:3, provided that said polypeptide is not TFPI; (7) a polypeptide comprising at least 3 (e.g. 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75 or more) amino acids from the C-terminus of amino acid sequence SEQ ID NO:1, provided that said polypeptide is not TFPI or a TFPI analog.

Antimicrobial activity has also been seen in peptides derived from the CTD, but not including the most C-terminal residues of TFPI. Thus the invention provides a polypeptide comprising a fragment of at least 3 (e.g. 4, 5, 6, 7, 8, 9, 10, 11, 12, 13, 14) consecutive amino acids of amino acid sequence SEQ ID NO: 5. The polypeptide may or may not itself be a fragment of TFPI (e.g. of SEQ ID NO: 1) or a TFPI analog.

The invention also provides a polypeptide comprising a fragment of at least 3 (e.g. 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71,72, 73, 74, 75, 76, 77, 78, 79, 80, 81, 82, 83, 84, 85, 86, 87, 88, 89, 90, 91, 92, 93, 94, 95, 96, 97, 98, 99, 100, or more) consecutive amino acids of amino acid sequence SEQ ID NO: 6. The polypeptide may or may not itself be a fragment of TFPI (e.g. of SEQ ID NO: 1). Preferred fragments of SEQ ID NO:6 are also fragments of SEQ ID NO: 5.

The invention also provides a polypeptide comprising a fragment of SEQ ID NO: 1, provided that (a) the fragment includes at least 3 (e.g. 4, 5, 6, 7, 8, 9, 10, 11, 12, 13, 14) consecutive amino acids of amino acid sequence SEQ ID NO: 5, and (b) the polypeptide is not TFPI or a TFPI analog.

The invention also provides: (1) a polypeptide consisting of amino acid sequence SEQ ID NO:7 (peptide #3); (2) a polypeptide comprising amino acid sequence SEQ ID NO:7, provided that the polypeptide is not TFPI or a TFPI analog; (3) a polypeptide comprising amino acid sequence SEQ ID NO:7, provided that the amino acid (if one is present) to the N-terminus of SEQ ID NO:7 is not Lys; (4) a polypeptide comprising an amino acid sequence that is at least 50% (e.g. ≧60%, ≧70%, ≧80%, ≧85%, ≧90%, ≧92%, ≧94%, ≧96%, ≧98%, or more) identical to SEQ ID NO: 7; (5) a polypeptide comprising amino acid sequence SEQ ID NO:7, provided that at least one of the amino acids in said SEQ ID NO:7 is a D-amino acid; (6) a polypeptide comprising a fragment of at least 3 (e.g. 4, 5, 6, 7, 8, 9, 10, 11, 12, 13, 14, 15, 16, 17, 18, 19, 20 or 21) consecutive amino acids of amino acid sequence SEQ ID NO:7, provided that said polypeptide is not TFPI or a TFPI analog.

The invention also provides: (1) a polypeptide consisting of amino acid sequence SEQ ID NO:5; (2) a polypeptide comprising amino acid sequence SEQ ID NO:5, provided that the polypeptide is not TFPI or a TFPI analog; (3) a polypeptide comprising amino acid sequence SEQ ID NO:5, provided that the amino acid (if one is present) to the N-terminus of SEQ ID NO:5 is not Lys; (4) a polypeptide comprising an amino acid sequence that is at least 50% (e.g. ≧60%, ≧70%, ≧80%, ≧85%, ≧90%, ≧92%, ≧94%, ≧96%, ≧98%, or more) identical to SEQ ID NO: 5; (5) a polypeptide comprising amino acid sequence SEQ ID NO:5, provided that at least one of the amino acids in said SEQ ID NO:5 is a D-amino acid; (6) a polypeptide comprising a fragment of at least 3 (e.g. 4, 5, 6, 7, 8, 9, 10, 11, 12, 13, 14, 15, 16, 17, 18, 19, 20 or 21) consecutive amino acids of amino acid sequence SEQ ID NO:5, provided that said polypeptide is not TFPI or a TFPI analog.

The invention also provides: (1) a polypeptide consisting of amino acid sequence SEQ ID NO:10 (peptide #5); (2) a polypeptide comprising amino acid sequence SEQ ID NO:10, provided that the polypeptide is not TFPI or a TFPI analog; (3) a polypeptide comprising amino acid sequence SEQ ID NO:10, provided that the amino acid (if one is present) to the N-terminus of SEQ ID NO:10 is not Lys; (4) a polypeptide comprising an amino acid sequence that is at least 50% (e.g. ≧60%, ≧70%, ≧80%, ≧85%, ≧90%, ≧92%, ≧94%, ≧96%, ≧98%, or more) identical to SEQ ID NO: 10; (5) a polypeptide comprising amino acid sequence SEQ ID NO:10, provided that at least one of the amino acids in said SEQ ID NO:10 is a D-amino acid; (6) a polypeptide comprising a fragment of at least 3 (e.g. 4, 5, 6, 7, 8, 9, 10, 11, 12, 13, 14, 15, 16, 17, 18, 19, 20 or 21) consecutive amino acids of amino acid sequence SEQ ID NO:10, provided that said polypeptide is not TFPI or a TFPI analog.

These polypeptides can preferably bind to LPS and/or to bacteria. These polypeptides may also bind to mannoproteins found in the cell wall of pathogenic fungi. The polypeptides may also bind to proteins found on viral particles.

The polypeptides preferably consist of no more than 250 amino acids (e.g. no more than 225, 200, 190, 180, 170, 160, 150, 140, 130, 120, 110, 100, 95, 90, 80, 70, 60, 50, 45, 40, 35, 30, 25, 20, 19, 18, 17, 16, 15, 14, 13, 12, 11, 10, 9, 8, 7, 6 or even 5 amino acids). Polypeptides consisting of between 5 and 90 amino acids are preferred (e.g. consisting of between 5 and 80, 5 and 70, 5 and 60 amino acids, etc.). Particularly preferred are polypeptides consisting of between 8 and 25 amino acids.

The polypeptide preferably consists of at least 3 amino acids (e.g. at least 4, 5, 6, 7, 8, 9, 10, 11, 12, 13, 14, 15, 16, 17, 18, 19, 20, 25, 30, 35, 40, 45, or at least 50 amino acids).

The invention provides a polypeptide having formula NH₂-A-B-C-COOH, wherein: A is a polypeptide sequence consisting of a amino acids; C is a polypeptide sequence consisting of c amino acids; B is a polypeptide sequence which is a fragment of at least b consecutive amino acids from the amino acid sequence SEQ ID NO:3, where b is 3 or more (e.g. 4, 5, 6, 7, 8, 9, 10, 11, 12, 13, 14, 15, 16, 17, 18, 19, 20 or 21).

The invention provides a polypeptide having formula NH₂-A-B-C-COOH, wherein: A is a polypeptide sequence consisting of a amino acids; C is a polypeptide sequence consisting of c amino acids; B is a polypeptide sequence which is a fragment of at least b consecutive amino acids from the amino acid sequence SEQ ID NO:5, wherein b is 3 or more (e.g. 4, 5, 6, 7, 8, 9, 10, 11, 12, 13, 14).

The invention provides a polypeptide having formula NH₂-A-B-C-COOH, wherein: A is a polypeptide sequence consisting of a amino acids; C is a polypeptide sequence consisting of c amino acids; B is a polypeptide sequence which is a fragment of at least b consecutive amino acids from the amino acid sequence SEQ ID NO:7, wherein b is 3 or more (e.g. 4, 5, 6, 7, 8, 9, 10, 11, 12, 13, 14).

The invention provides a polypeptide having formula NH₂-A-B-C-COOH, wherein: A is a polypeptide sequence consisting of a amino acids; C is a polypeptide sequence consisting of c amino acids; B is a polypeptide sequence which is a fragment of at least b consecutive amino acids from the amino acid sequence SEQ ID NO:10, wherein b is 3 or more (e.g. 4, 5, 6, 7, 8, 9, 10, 11, 12, 13, 14).

The value of a is generally at least 1 (e.g. at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, 100, 150, 200, 250, 300, 350, 400, 450, 500 etc.). The value of c is generally at least 1 (e.g. at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, 100, 150, 200, 250, 300, 350, 400, 450, 500 etc.). The value of a+c is at least 1 (e.g. at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, 100, 150, 200, 250, 300, 350, 400, 450, 500 etc.). It is preferred that the value of a+c is at most 1000 (e.g. at most 900, 800, 700, 600, 500, 450, 400, 350, 300, 250, 200, 190, 180, 170, 160, 150, 140, 130, 120, 110, 100, 90, 80, 70, 60, 50, 40, 30, 25, 20, 19, 18, 17, 16, 15, 14, 13, 12, 11, 10, 9, 8, 7, 6, 5, 4, 3, 2).

The amino acid sequence of -A- typically shares less than m % sequence identity to the a amino acids which are N-terminal of sequence -B- in SEQ ID NO:2. The amino acid sequence of -C- typically shares less than n % sequence identity to the c amino acids which are C-terminal of sequence -B- in SEQ ID NO:2 variable region of an antibody of the invention (e.g. in SEQ ID NO: 2). In general, the values of m and n are both 60 or less (e.g. 50, 40, 30, 20, 10 or less). The values of m and n may be the same as or different from each other.

In some embodiments of the invention, the polypeptides do not consist of SEQ ID NO:4, which was disclosed by Hembrough et al. in reference 10 as having anti-tumor and anti-angiogenic activity, but not as having anti-bacterial activity.

Polypeptides of the invention may comprise amino acid sequences that have sequence identity to SEQ ID NO: 3, 5, 6, 7 and 10. These polypeptides include homologs, orthologs, allelic variants and mutants. Identity between polypeptides is preferably determined by the Smith-Waterman homology search algorithm as implemented in the MPSRCH program (Oxford Molecular), using an affine gap search with parameters gap open penalty=12 and gap extension penalty=1.

These polypeptides may, compared to SEQ ID NO 3, 5, 6, 7 and 10, include one or more (e.g. 1, 2, 3, 4, 5, 6, etc.) conservative amino acid substitutions i.e. replacements of one amino acid with another which has a related side chain. Genetically encoded amino acids are generally divided into four families: (1) acidic i.e. aspartate, glutamate; (2) basic i.e. lysine, arginine, histidine; (3) non-polar i.e. alanine, valine, leucine, isoleucine, proline, phenylalanine, methionine, tryptophan; and (4) uncharged polar i.e. glycine, asparagine, glutamine, cysteine, serine, threonine, tyrosine. Phenylalanine, tryptophan, and tyrosine are sometimes classified jointly as aromatic amino acids. In general, substitution of single amino acids within these families does not have a major effect on the biological activity. Moreover, the polypeptides may have one or more (e.g. 1, 2, 3, 4, 5, 6 etc.) single amino acid deletions relative to a reference sequence. Furthermore, the polypeptides may include one or more (e.g. 1, 2, 3, 4, 5, 6 etc.) insertions (e.g. each of 1, 2 or 3 amino acids) relative to a reference sequence.

Polypeptides of the invention can be prepared in many ways e.g. by chemical synthesis (in whole or in part), by digesting TFPI using proteases, by translation from RNA, by purification from cell culture (e.g. from recombinant expression), etc. A preferred method for production of peptides <40 amino acids long involves in vitro chemical synthesis [11,12]. Solid-phase peptide synthesis is particularly preferred, such as methods based on tBoc or Fmoc [13] chemistry. Enzymatic synthesis [14] may also be used in part or in full. As an alternative to chemical synthesis, biological synthesis may be used e.g. the polypeptides may be produced by translation. This may be carried out in vitro or in vivo. Biological methods are in general restricted to the production of polypeptides based on L-amino acids, but manipulation of translation machinery (e.g. of aminoacyl tRNA molecules) can be used to allow the introduction of D-amino acids (or of other non natural amino acids, such as iodotyrosine or methylphenylalanine, azidohomoalanine, etc.) [15]. Where D-amino acids are included, however, it is preferred to use chemical synthesis. Polypeptides of the invention may have covalent modifications at the C-terminus and/or N-terminus.

Polypeptides of the invention can take various forms (e.g. native, fusions, glycosylated, non-glycosylated, lipidated, non-lipidated, phosphorylated, non-phosphorylated, myristoylated, non-myristoylated, monomeric, multimeric, particulate, denatured, etc.).

Polypeptides of the invention are preferably provided in purified or substantially purified form i.e. substantially free from other polypeptides (e.g. free from naturally-occurring polypeptides), and are generally at least about 50% pure (by weight), and usually at least about 90% pure i.e. less than about 50%, and more preferably less than about 10% (e.g. 5% or less) of a composition is made up of other expressed polypeptides.

Polypeptides of the invention may be attached to a solid support. Polypeptides of the invention may comprise a detectable label (e.g. a radioactive or fluorescent label, or a biotin label).

The term “polypeptide” refers to amino acid polymers of any length. The polymer may be linear, branched or circular, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. Polypeptides can occur as single chains or associated chains.

The invention provides polypeptides comprising one or more sequences -X-Y- or -Y-X- or -X-X-, wherein: -X- is an amino acid sequence as defined above and -Y- is not a sequence as defined above i.e. the invention provides fusion proteins. For example, the invention provides -X₁-Y₁-X₂-Y₂- , or X₁-X₂-Y₁ or -X₁-X₂- etc. In one embodiment of the invention, Y is an N-terminal leader sequence as seen for example in SEQ ID No 14 or 15. In a further embodiment, Y is a C-terminal T-helper sequence as seen for example in SEQ ID No 16 or 17.

The invention provides a process for producing polypeptides of the invention, comprising the step of culturing a host cell of to the invention under conditions that induce polypeptide expression.

The invention provides a process for producing a polypeptide of the invention, wherein the polypeptide is synthesised in part or in whole using chemical means.

Combination of C-terminus Polypeptides With TFPI

TFPI has an anti-coagulant effect and it also interrupts potentially harmful endotoxin signaling. In addition, as described herein, it has an anti-microbial effect, for example an anti-bacterial effect, mediated by its C-terminus domain. To enhance the anti-bacterial effect of TFPI and TFPI analogs, TFPI (or a TFPI analog) may be administered in conjunction with a polypeptide, as defined above, from the C-terminus of TFPI. Alternatively, or in addition, to enhance the anti-microbial effect of the polypeptides as defined above, from the C-terminus region of TFPI, they may be administered in conjunction with TFPI and/or a TFPI analog .

Thus the invention provides: (1) a pharmaceutical composition comprising TFPI, or a TFPI analog, and an anti-microbial polypeptide of the invention; (2) TFPI or a TFPI analog, and an anti-microbial polypeptide of the invention, for simultaneous separate or sequential administration; (3) a method for treating a patient comprising simultaneous separate or sequential administration of TFPI, or a TFPI analog, and an anti-microbial polypeptide of the invention; (4) a method for treating a patient comprising administration of TFPI, or a TFPI analog, to a patient who has received an anti-microbial polypeptide of the invention; (4) a method for treating a patient comprising administration of an anti-microbial polypeptide of the invention to a patient who has received TFPI, or a TFPI analog. The anti-microbial is preferably anti-bacterial.

Thus the invention provides: (1) a pharmaceutical composition comprising an anti-microbial polypeptide of the invention and TFPI or a TFPI analog, and (2) an anti-microbial compound of the invention and TFPI or a TFPI analog, for simultaneous separate or sequential administration; (3) a method for treating a patient comprising simultaneous separate or sequential administration of an anti-microbial compound of the invention and TFPI or a TFPI analog, and (4) a method for treating a patient comprising administration of TFPI or a TFPI analog, to a patient who has received an anti-microbial compound of the invention; (4) a method for treating a patient comprising administration of an anti-microbial compound of the invention to a patient who has received TFPI or a TFPI analog.

The TFPI analog used in these combinations may include, or alternatively may lack, the C-terminus-derived anti-microbial polypeptide or anti-bacterial polypeptide. Thus the TFPI may lack up to q C-terminus amino acids, as described above.

Drug Design and Peptidomimetics

Polypeptides of the invention are useful anti-microbials in their own right. However, they may be refined to improve anti-microbial activity (either general or specific) or to improve pharmacologically important features such as bio-availability, toxicology, metabolism, pharmacokinetics etc. The polypeptides may therefore be used as lead compounds for further research and refinement.

Polypeptides of the invention can be used for designing peptidomimetic molecules [16-21]. Peptidomimetic techniques have successfully been used to design thrombin inhibitors [22,23]. These will typically be isosteric with respect to the polypeptides of the invention but will lack one or more of their peptide bonds. For example, the peptide backbone may be replaced by a non-peptide backbone while retaining important amino acid side chains. The peptidomimetic molecule may comprise sugar amino acids [24]. Peptoids may be used.

To assist in the design of peptidomimetic molecules, a pharmacophore (ie. a collection of chemical features and 3D constraints that expresses specific characteristics responsible for activity) can be defined for the peptides. The pharmacophore preferably includes surface-accessible features, more preferably including hydrogen bond donors and acceptors, charged/ionisable groups, and/or hydrophobic patches. These may be weighted depending on their relative importance in conferring activity [25].

Pharmacophores can be determined using software such as CATALYST (including HypoGen or HipHop), CERIUS², or constructed by hand from a known conformation of a polypeptide of the invention. The pharmacophore can be used to screen structural libraries, using a program such as CATALYST. The CLIX program can also be used, which searches for orientations of candidate molecules in structural databases that yield maximum spatial coincidence with chemical groups which interact with the receptor.

The binding surface or pharmacophore can be used to map favourable interaction positions for functional groups (e.g. protons, hydroxyl groups, amine groups, hydrophobic groups) or small molecule fragments. Compounds can then be designed de novo in which the relevant functional groups are located in substantially the same spatial relationship as in polypeptides of the invention.

Functional groups can be linked in a single compound using either bridging fragments with the correct size and geometry or frameworks which can support the functional groups at favourable orientations, thereby providing a peptidomimetic compound according to the invention. Whilst linking of functional groups in this way can be done manually, perhaps with the help of software such as QUANTA or SYBYL, automated or semi-automated de nova design approaches are also available, such as:
MCSS/HOOK [26, 27], which links multiple functional groups with molecular templates taken from a database.
LUDI [28], which computes the points of interaction that would ideally be fulfilled by a ligand, places fragments in the binding site based on their ability to interact with the receptor, and then connects them to produce a ligand.
MCDLNG [29], which fills a receptor binding site with a close-packed array of generic atoms and uses a Monte Carlo procedure to randomly vary atom types, positions, bonding arrangements and other properties.
GROW [30], which starts with an initial ‘seed’ fragment (placed manually or automatically) and grows the ligand outwards.
SPROUT [31], suite which includes modules to: identify favourable hydrogen bonding and hydrophobic regions within a binding pocket (HIPPO module); select functional groups and position them at target sites to form starting fragments for structure generation (EleFAnT); generate skeletons that satisfy the steric constraints of the binding pocket by growing spacer fragments onto the start fragments and then connecting the resulting part skeletons (SPIDeR); substitute hetero atoms into the skeletons to generate molecules with the electrostatic properties that are complementary to those of the receptor site (MARABOU). The solutions can be clustered and scored using the ALLigaTOR module.
CAVEAT [32], which designs linking units to constrain acyclic molecules.
LEAPFROG [33], which evaluates ligands by making small stepwise structural changes and rapidly evaluating the binding energy of the new compound. Changes are kept or discarded based on the altered binding energy, and structures evolve to increase the interaction energy with the receptor.
GROUPBUILD [34], which uses a library of common organic templates and a complete empirical force field description of the non-bonding interactions between a ligand and receptor to construct ligands that have chemically reasonable structure and have steric and electrostatic properties complimentary to the receptor binding site.
RASSE [35]

These methods identify relevant compounds. These compounds may be designed de novo, may be known compounds, or may be based on known compounds. The compounds may be useful themselves, or they may be prototypes which can be used for further pharmaceutical refinement (i.e. lead compounds) in order to improve binding affinity or other pharmacologically important features (e.g. bio-availability, toxicology, metabolism, pharmacokinetics etc.).

As well as being useful compounds individually, peptidomimetics identified in silico by the structure-based design techniques can also be used to suggest libraries of compounds for ‘traditional’ in vitro or in vivo screening methods. Important pharmaceutical motifs in the ligands can be identified and mimicked in compound libraries (e.g. combinatorial libraries) for screening for anti-microbial activity.

The invention provides: (i) a compound identified using these drug design methods; (ii) a compound identified using these drug design methods, for use as a pharmaceutical; (iii) the use of a compound identified using these drug design methods in the manufacture of an anti-microbial such as an anti-bacterial; and (iv) a method of treating a patient with a microbial, such as, bacterial infection, comprising administering an effective amount of a compound identified using these drug design methods.

Therapeutic Methods and Compositions

The invention provides compositions comprising: (a) compounds, polypeptides, and/or peptidomimetics of the invention; and (b) a pharmaceutically acceptable carrier. The compositions of the invention are useful to treat patients at risk of developing, or diagnosed as having, a microbial infection or to lower the risk of the infection developing into a severe infection for one or a group of patients.

Component (a) is the active ingredient in the composition, and this is present at a therapeutically effective amount i.e. an amount sufficient to inhibit microbial growth and/or survival in a patient, and preferably an amount sufficient to eliminate microbial infection. The precise effective amount for a given patient will depend upon their size and health, the nature and extent of infection, and the composition or combination of compositions selected for administration. The effective amount can be determined by routine experimentation and is within the judgment of the clinician. For purposes of the present invention, an effective dose will generally be from about 0.01 mg/kg to about 5 mg/kg, or about 0.01 mg/kg to about 50 mg/kg or about 0.05 mg/kg to about 10 mg/kg. Pharmaceutical compositions based on polypeptides are well known in the art. Polypeptides may be included in the composition in the form of salts and/or esters.

A ‘pharmaceutically acceptable carrier’ includes any carrier that does not itself induce the production of antibodies harmful to the individual receiving the composition. Suitable carriers are typically large, slowly metabolised macromolecules such as proteins, polysaccharides, polylactic acids, polyglycolic acids, polymeric amino acids, amino acid copolymers, sucrose, trehalose, lactose, and lipid aggregates (such as oil droplets or liposomes). Such carriers are well known to those of ordinary skill in the art.

The pharmaceutical composition may be administered by means known in the art. This can include, but is not limited to, topical application and intravenous, aerosol, subcutaneous, and intramuscular routes. The pharmaceutical composition can be given as a single dose or in multiple doses.

The microbial infection may be with a single microbial species, or with several microbes. It may be a combination of infection by any two or more of bacteria, virus, or fungi. When the microbial infection results from bacteria, it may be with a Gram-positive bacterium and/or a Gram-negative bacterium. Typical Gram-negative bacteria involved in severe infections include Escherichia coli, Bacteroides fragilis, Pseudomonas aeruginosa, Klebsiella species, Enterobacter species, and Proteus species. Typical Gram-positive bacteria involved in severe infections include Streptococcus pneumoniae, Staphylococcus aureus, Staphylococcus epidermidis, Enterococcus species, Streptococcus agalactiae and Streptococcus pyogenes. Severe fungal infections may involve Candida albicans, Candida glabrata, Aspergillus fumigatus, Aspergillus niger, Cryptococcus neoformans and Fusarium species. Viral infections can be associated with human immunodeficiency virus (HIV), herpes simplex, human papilloma virus, hepatitis virus, reovirus, adenovirus, influenza, and human T-cell leukemia virus. Parasitic protozoal infections can be associated with Trypanosoma cruzi, and Leishmania, Giardia, Entamoeba and Plasmodium species.

Microbial infections include, for example, pneumonia, ear infections, diarrhea, urinary tract infections, skin disorders, topical and mucosal as well as disseminated invasive fungal infections.

Compositions of the invention may include an additional antimicrobial, particularly if packaged in a multiple dose format.

The invention also provides the use of the compounds and polypeptides of the invention in the manufacture of a medicament for treating a patient at risk of developing or diagnosed as having a microbial infection.

Preferred patients for treatment are human, including children (e.g. a toddler or infant), teenagers and adults.

General

The term “comprising” encompasses “including” as well as “consisting” e.g. a composition “comprising” X may consist exclusively of X or may include something additional e.g. X+Y.

The term “about” in relation to a numerical value x means, for example, x±10%. Where necessary, the term “about” can be omitted.

The word “substantially” does not exclude “completely” e.g. a composition which is “substantially free” from Y may be completely free from Y. Where necessary, the word “substantially” may be omitted from the definition of the invention.

Percent sequence identity can be determined using the Smith-Waterman homology search algorithm using an affine gap search with a gap open penalty of 12 and a gap extension penalty of 2, BLOSUM matrix of 62. The Smith-Waterman homology search algorithm is taught in ref. 36.

As indicated in the above text, nucleic acids and polypeptides of the invention may include sequences that:
(a) are identical (i.e. 100% identical) to the sequences disclosed in the sequence listing;
(b) share sequence identity with the sequences disclosed in the sequence listing;
(c) have 1, 2, 3, 4, 5, 6, 7, 8, 9 or 10 single nucleotide or amino acid alterations (deletions, insertions, substitutions), which may be at separate locations or may be contiguous, as compared to the sequences of (a) or (b); and
(d) when aligned with a particular sequence from the sequence listing using a pairwise alignment algorithm, a moving window of x monomers (amino acids or nucleotides) moving from start (N-terminus or 5′) to end (C-terminus or 3′), such that for an alignment that extends to p monomers (where p>x) there are p−x+1 such windows, each window has at least x·y identical aligned monomers, where: x is selected from 5, 6, 7, 8, 9, 10, 11, 12, 13, 14, 15, 16, 17, 18, 19, 20, 21, 22, 23, 24, 25, 26, 27, 28, 29, 30, y is selected from 0.50, 0.60, 0.70, 0.75, 0.80, 0.85, 0.90, 0.91, 0.92, 0.93, 0.94, 0.95, 0.96, 0.97, 0.98, 0.99; and if x·y is is not an integer then it is rounded up to the nearest integer. The preferred pairwise alignment algorithm is the Needleman-Wunsch global alignment algorithm [37], using default parameters (e.g. with Gap opening penalty=10.0, and with Gap extension penalty=0.5, using the EBLOSUM62 scoring matrix). This algorithm is conveniently implemented in the needle tool in the EMBOSS package [38].

The nucleic acids and polypeptides of the invention may additionally have further sequences to the N-terminus/5′ and/or C-terminus/3′ of these sequences (a) to (d).

The terms “microbial” and “microorganism” encompass all microbes including bacteria, viruses and fungi.

The term “animal” refers to any member of the animal kingdom including human beings. Compounds of the invention are useful in animals that have a complement system.

The practice of the present invention will employ, unless otherwise indicated, conventional methods of chemistry, biochemistry, molecular biology, immunology and pharmacology, within the skill of the art. Such techniques are explained fully in the literature, e.g., see references 39-46, etc.

The complement system is a biochemical cascade of the immune system that helps clear microbial pathogens from an organism by disrupting the target cell's plasma membrane or by increasing opsonization. The classical complement pathway is triggered by activation of the C1-complex (composed of C1q, C1r and C1s). Activation can involve conformational changes in C1q molecule, which leads to the activation of C1r serine protease molecules and subsequence cleavage of C1s. The resulting C1-complex can bind to C2 and C4, producing C2b and C4b by cleavage. The alternative complement pathway is triggered by C3 hydrolysis directly on the surface of a pathogen. The lectin complement pathway is homologous to the classical pathway, but with the opsonin, mannan-binding lectin (MBL) and ficolins, instead of C1q. The cytolytic end product of complement is the membrane attack complex (MAC), consisting of C5b, C6, C7, C8, and C9. The MAC forms a transmembrane channel, which causes osmotic lysis of the target cell. Compounds of the invention may interact with complement, or a component thereof such as C1q.

### BRIEF DESCRIPTION OF DRAWINGS

FIG. 1 shows bacterial killing activity of proteolytically digested TFPI in whole blood. TFPI was proteolyzed with plasmin, thrombin (1A), elastase (1B) or cathepsin G (1C). TFPI proteolyzed with cathepsin G showed the highest antibacterial activity in comparison to either cathepsin G alone or whole TFPI.

FIG. 2 shows that increasing the incubation time of proteolysis results in increased anti-bacterial activity. 2A shows an SDS-PAGE depiction of the proteolytic digest over time while 2B shows the increase in anti-bacterial activity of the digests. 2C demonstrates that the killing is not due to cathepsin G.

FIG. 3 shows the major protolytic fragments generated by digestion. TFPI was digested with cathepsin G and gel filtration fractions collected.

FIG. 4 demonstrates the anti-bacterial activity of the gel filtration fractions following the TFPI proteolysis.

FIG. 5 depicts the gel filtration fractions when the gel filtration is performed in the presence of 1 M NaCl.

FIG. 6 depicts the 1 M NaCl gel filtration fractions (6A) after desalting, and demonstrates the recovery of the anti-bacterial activity from the column (6B). 6C shows the anti-bacterial activity of the purified TFPI fragments shown in 6A.

FIG. 7 shows the peptides selected for N-terminal sequencing and mass spectrometry analysis from the bands in the gel filtration fractions with anti-bacterial activity.

FIG. 8 shows that the anti-bacterial activity is dependent upon active complement. 8A and 8B demonstrate that killing activity is decreased upon heat inactivation or either plasma (A) or serum (B). 8C demonstrates that the killing activity can also be lessened by treatment with cobra venom factor. 8D demonstrates that the killing activity of peptide in serum is similar to that in whole blood.

FIG. 9 demonstrates that the classical complement pathway is essential for the anti-bacterial activity of the TFPI peptides.

FIG. 10 depicts the binding of fluorescently labeled peptide to bacteria, and demonstrates that the binding may be inhibited by the presence of heparin. FIGS. 10A-10D are 1000×, fluorescent; FIGS. 10E-10H are 1000×, white light.

FIG. 11 shows the importance of the C-terminal region of TFPI for the anti-bacterial activity of TFPI.

### EXAMPLES

The present invention will now be illustrated by reference to the following examples that set forth particularly advantageous embodiments. However, it should be noted that these embodiments are illustrative and are not to be construed as restricting the invention in any way.

In the examples that follow, all exogenous TFPI is the TFPI analog, ala-TFPI.

### Example 1

### Antibacterial Effects of Proteolyzed TFPI

Experiment I

TFPI was incubated with α-thrombin, plasmin, elastase and cathepsin G in 10% blood and tested for bacterial killing activity against E. coli O18:K1:H7 as follows. Recombinant TFPI at 5 μM (unless indicated otherwise), was treated with plasmin (100 nM, lane 3) and α-thrombin (100 nM, lane 7; 1 μM, lane 8) (A), elastase (100 nM, lanes 3 and 4) (B), and cathepsin G (100 nM, lane 4; 1 μM, lanes 5, 6 and 7) (C) in a total volume of 180 μl. The reaction contained TFPI diluted in RPMI 1640 without phenol red, 25 mM Hepes, pH 7.5 (RPMI), 3000 CFU E. coli O18:K1:H7 in 40 μl PBS, and diluted enzyme in 20 μl RPMI. The volume was adjusted with RPMI. The samples were pre-incubated for 15-30 minutes before the addition of anti-coagulant-free fresh blood to a final volume of 200 μl. Controls were 10% blood in RPMI, TFPI at 1, 2 and 5 μM, and the enzymes alone at the respective concentrations. The samples were incubated for 4 hours at 37° C. with 5% CO₂ in a humidified incubator and serial dilutions plated on Tryptase soy agar plates. The number of bacterial colonies was determined after overnight incubation at 37° C. All experimental conditions are run in duplicate. The data represent the average colony number.

Experiment II

TFPI was proteolytically digested using cathepsin G and tested for bacterial killing activity against E. coli O18:K1:H7 in the presence of 10% blood as follows. TFPI (1.2 mg) at 10 mg/ml in formulation buffer (300 mM L-arginine, 5 mM methionine, 20 mM Na-citrate, pH 5.5) was digested with cathepsin G (in 150 mM NaCl, 50 mM Na-acetate, pH 5.5) at 10 μM and 1 μl and 18 μl aliquots were taken over 5 days. The 1 μl aliquots were analysed by SDS-PAGE gel electrophoresis on 10-20% glycine gels.

As seen in FIG. 2A, incubation of TFPI with cathepsin G over 5 days resulted in partial digestion (M, Prestained Standard; S, start of digestion).

The 18 μl aliquots were diluted to 10 μM TFPI with 445 μl RPMI 1640 without phenolred, 25 mM Hepes, pH 7.5 (RPMI) and assayed at a final concentration of 5 μM in 200 μl reactions containing 3000 CFU E. coli in 40 μl PBS, 10% non-coagulated blood, and adjusted to the final volume with RPMI. Negative controls were 10% blood in RPMI, TFPI at 5 μM and cathepsin G at 400 nM. Anti-coagulant free blood was freshly drawn and added to the reaction as the last component. The samples were manipulated as described above.

As seen in FIGS. 1 and 2, digested TFPI interfered with the bacterial growth, with an increase of activity over time. Cathepsin G and undigested TFPI did not exhibit a similar activity. Thus, fragmentation of TFPI results in the release of a novel activity.

Experiment III

To define the active regions of the TFPI proteolytic fragments, TFPI was digested at preparative scale and subjected to gel filtration in RPMI as follows. TFPI (12 mg or 23 mg) was digested with cathepsin G as before for 2 days and parallel samples fractionated by gel filtration over a Hiload Superdex 30 16/60 column in RPMI, or alternatively in RPMI with NaCl added as solid to a final concentration of 1M. Fractions of 1 ml were collected and analysed by SDS-PAGE gel electyrophoresis on 16% tricine gels.

FIGS. 3 and 5 show the separation achieved in RPMI and RPMI with 1M NaCl, respectively. As can be seen by comparing FIG. 3 and FIG. 5, an improved separation of fragment 1 from fragment 3 is achieved in 1 M NaCl and fragment 2 is eluted in a defined number of fractions in 1M NaCl, only.

The fractions were tested for bacterial killing activity as follows: Aliquots of 100 μl of the fractions derived from gel filtration in RPMI were directly added to reactions of 200 μl as above and the effect on the outgrowth of bacterial colonies assayed. The fractions containing 1M NaCl were desalted in RPMI to about 155 mM NaCl and a fraction size of 600 μl using Centrifugal Devices with the cut-off of 1 KDa. Shown in FIG. 6A are the fractions derived from gel filtration in 1M NaCl. Aliquots of 25 μl or 100 μl of the fractions were added to reactions of 200 μl as above to assay for an effect on bacterial survival.

As can be seen in FIG. 4, after gel filtration in RPMI all fractions assayed exert only minor activity in the bacterial killing assay. In contrast, as shown in FIG. 6B, several of the fractions derived from gel filtration in 1M NaCl exhibit strong bacterial killing activity. The highest activity is found in fractions 21 and 22. Fractions 27 and 28 showed weak activity when added at a higher concentration (4-fold increased fraction volume). This data demonstrates that digested TFPI contains antibacterial activity.

Experiment IV

Fragments 1, 2 and 3 identified in FIG. 6A were further purified. Briefly, Mono-S columns were used for cation exchange with a 0.5 M-1 M NaCl-gradient in 50 mM Hepes, pH 7 to purify fragments 1 and 2. A Mono-Q column was used for anion exchange with a 50 mM-1M NaCl-gradient for purification of fragment 3. Fractions of 1 ml were collected and analyzed by SDS-PAGE gel electrophoresis, as before. Purified fragments were then buffer-exchanged into RPMI, as before, and assayed for bacterial killing activity.

As shown in FIG. 6C, purified fragments from the C-terminus of TFPI have bacterial killing activity. Decreasing concentrations (1:2 dilutions) of fragment 1 (identified as aa161/165-276; lanes 1, 2, 3) show decreasing activity levels. Fragment 2 (identified as 183-269/276, lane 4) at a concentration similar to fragment 1 (lane 2, determined by comparative SDS-PAGE gel electrophoresis) exerts similar activity. Fragment 3 (identified as aa1-90) is without activity.

### Example 2

### N-terminal Sequencing of TFPI Proteolytic Fragments

To identify the molecular identity of the biologically active fragments, specific proteolytic bands from gelfiltration fractions 21, 23 and 25 were isolated and subjected to N-terminal sequencing as follows. Aliquots of the fractions were separated by SDS-PAGE electrophoresis on a 16% tricine gel and blotted onto a PVDF membrane in 10 mM CAPS, 10% methanol, (pH 11). The membrane was stained for 1 minute with 0.025% Coomassie Brilliant Blue G in 40% methanol, de-stained for 30 minutes with several changes of 50% methanol, and the bands of interest excised as indicated by boxes in FIG. 7. Mass spectroscopy was also performed on these fractions using LC-ESI-MS.

The results of the N-terminal sequence determination demonstrate that the fractions with anti-bacterial activity contain fragments derived from the C-terminal region of TFPI. Results were as follows: The major species identified by N-terminal sequencing in fraction 21 start with amino acid 161 and 165, in fraction 23 with amino acid 1, and in fraction 25 with amino 183. These results, and the species identified by mass spectrometry, are summarized below.

Taken together the results indicated that the major protein species in the active fractions are derived from the C-terminal domain of TFPI and include amino acids 161-269, amino acids 165-269, amino acids 183-276 and amino acids 183-269. A fragment derived from the N-terminus of TFPI (amino acids 1-90) was also found in the same fractions but is inactive.

| N-terminal-sequencing | | | | |
|---|---|---|---|---|
| Fraction | Start aa | Sequences | SEQ ID | Mass spectrometry |

| | | | | |
|---|---|---|---|---|
| 21 | 161 | GTQLNAVNNSLTPQS | 12 | 12329.9 Da [aa 161-269] |
| | 165 | NAVNNSLTPQSTKVX | 13 | 11929.2 Da [aa 165-269] |
| | | | | 10455.6 Da [aa 1-90] |
| 23 | 1 | ADSEEDEEHTIITDT | 14 | 10455.6 Da [aa 1-90] |
| | 161 | GTQLNAVNNSLXXXX | 15 | 12329.9 Da [161-269]; |
| | | | | 11930.7 Da [165-269] |
| | 165 | NXVNXXLTXXXXXXX | 16 | 10890.4 Da [183-276]; |
| | | | | 10030.1 Da [183-269] |
| 25 | 183 | EFHGPSWXLTPADRG | 17 | 10031.0 Da [183-269]; |
| | | | | 100891.3 Da [183-276] |
| | 1 | ADSEEDEXXXXXXXX | 18 | 10455.5 Da [aa 1-90]; |
| | | | | 11929.7 Da [165-269] |

### Example 3

### Importance of C-terminal Region of TFPI

The ability of TFPI to induce IL-6 secretion in the presence of LPS was tested using TFPI and TFPI analogs, including: (i) TFPI₁₋₁₆₁, having just residues 1-161 of TFPI; (ii) TFPI with mutant K1; (iii) TFPI with mutant K2; (iv) TFPI with mutants K1 and K2.

When diluted, freshly drawn whole blood is incubated with LPS derived from the cell wall of Gram-negative bacteria, a cytokine cascade is induced within a few hours. As shown in FIG. 11a, loss of the K2 domain or of amino acids 162-276 (a region including the C terminal domain) results in loss of the ability to induce IL-6 secretion, leading to the conclusion that the K2 domain and something in the C-terminal ⅓ of TFPI are essential to this activity.

FIG. 11b shows results of a similar experiment. The ability of ala-TFPI to induce IL-6 is closely mimicked by Des-K3-TFPI, which lacks only the K3 domain. In contrast, truncation of the C-terminus to leave 161aa or 252aa gives a molecule with an IL-6 induction profile similar to green fluorescent protein, the negative control. Thus the ability to induce IL-6 could involve amino acids downstream of residue 252, at the C-terminus of TFPI.

A peptide consisting of the 22 C-terminal amino acids of TFPI (i.e. SEQ ID NO:3) was tested in an IL-6 assay in the presence of LPS. As shown in FIG. 11c, inclusion of the peptide completely reversed the effect of LPS on cytokine production, in a peptide and LPS dose-dependent manner. Thus this peptide appears to be able to neutralize the endotoxin activity of LPS.

FIG. 11d shows the results of incubating the 22-mer with E. coli O18ac:K1:H7 (ATCC). An inoculum of live bacteria was added to diluted whole blood in the IL-6 induction assay. The 22-mer dose-dependently reduced bacterial survival, indicated by the suppression of the outgrowth of bacterial colonies. 300 nM of peptide killed all bacteria.

Thus, the 22-mer can neutralize LPS, and also has a direct bactericidal effect on live bacteria. These activities may be part of the innate immune response in defense against infection by bacteria. The assays elucidate an aspect of the mechanism of action of the TFPI molecule and indicate TFPI and its analogs as a molecule able to modulate the progression of bacterial infection by neutralizing endotoxin and preventing interaction with serum and cellular receptors. This activity may play an important role at an early stage of sepsis, or after release of endotoxins after treatment with antimicrobial agents. High serum levels of LPS have been associated with fatal outcome in patients with septic shock [47].

### Example 4

### Antibacterial Effects of TFPI C-terminal Peptides

Experiment I

Peptides were designed to test for bacterial killing activity localized in the C-terminal domain of TFPI. Activity was measured against Gram negative (E coli O18:K1:H7. ATCC 700973) bacteria in the presence of 10% blood as described above. Controls were RPMI with 10% blood and 100 nM Tifacogin. The biological activity was determined from the reduction of bacterial colonies, as above.

The antibacterial effects of the 22-mer (SEQ ID NO: 3; ‘peptide #1’) were compared to a scrambled control peptide (SEQ ID NO: 8; ‘peptide #2’) and to a fragment of TFPI having a N-terminus shifted 13 amino acids further upstream and a C-terminus shifted 8 amino acids upstream (i.e. SEQ ID NO: 7; ‘peptide #3’). Peptide #3 includes the thrombin cleavage site that is located upstream of SEQ ID NO: 3 in natural TFPI. The 14-mer overlap of peptides #1 and #3 is SEQ ID NO: 5. Peptide #5 (SEQ ID NO. 10) includes the amino acids of peptide #3 and additionally the C-terminus 8 amino acids of peptide #1. The sequences and their corresponding peptide numbers are shown below:

| | | | |
|---|---|---|---|
| Peptide | Sequence | TFPI sequence | SEQ ID NO |

| | | | |
|---|---|---|---|
| #1 | TKRKRKKQRVKIAYEEIFVKNM | aas 255-276 | 3 |
| #2 | NFQRKEKREVIYKVKTKIKAMR | aas 255-276 scramble | 8 |
| #3 | GFIQRISKGGLIKTKRKRKKQRVKIAY | aas 242-268 | 7 |
| #4 | LLGDFFRKSKEKIGKEFKRIVQRIKDFLRNLVPRTES | LL-37, cap18 | 9 |
| #5 | GFIQRISKGGLIKTKRKRKKQRVKIAYEEIFVKNM | aas 242-276 | 10 |

The three peptides #1, #2, & #3 were diluted in H₂O to 10 fold their final assay concentration and incubated with bacteria for 4 hours, at the final concentrations of 3 μM, 300 nM 100 nM and 10 nM. E. coli was used at 3000 CFU/200 μl. Surviving colony numbers were determined as described in Example 1. As shown in Table 1, peptides #1 and #3 were both active against an O18ac:K1:H7 E. coli strain. Peptide #3 showed better activity than peptide #1, giving total killing of bacteria when incubated at 3000 nM with blood, suggesting that cleavage at the thrombin cleavage site is inactivating. Full-length TFPI has no activity against Gram negative bacteria in these assays at the concentrations tested. Table 2 shows biological activity of peptide #3 and peptide #5 after serial dilution to 300 nM, 100 nM and 10 nM. The activity of peptide #5 is similar, but slightly reduced, compared to peptide #3.

TABLE 1

| | Controls | | Peptide #1 | | Peptide #2 | | Peptide #3 | | Peptide #4 | |
|---|---|---|---|---|---|---|---|---|---|---|
| | Blood | TFPI | 3 μM | 300 nM | 3 μM | 300 nM | 3 μM | 300 nM | 3 μM | 300 nM |
| CFU | 5.1 | 5.2 | 1.1 | 5.2 | 5.4 | 5.3 | N.D. | 2.2 | 1.3 | 4.9 |
| (range) | (4.8-5.2) | | (0.4-1.2) | | (5.3-5.6) | (5.2-5.3) | | (1.4-2.4) | (1.3-1.4) | (4.4-5.1) |

N.D. = not detectable

TABLE 2

| | Peptide #3 | | | | Peptide #5 | | |
|---|---|---|---|---|---|---|---|
| | Blood | 300 nM | 100 nM | 10 nM | 300 nM | 100 nM | 10 nM |
| CFU | 3.5 | N.D. | 1.2 | 2.6 | N.D. | 2.1 | 4.1 |
| (range) | (3.4-3.6) | | (N.D.-1.4) | (2.2-3.1) | | (1.3-2.2) | (3.9-4.1) |

N.D. = not detectable

Experiment II

Peptide #3 was tested for activity at 3 μM on additional strains of E. coli using normal human serum as a control. As shown in Table 3, E. coli O2a, 2b:K5(L):H4 (ATCC 23500) and O7:K1(L):NM (ATCC 23503) were affected by peptide #3, in a similar manner to E. coli O18:K1:H7.

TABLE 3

| | O18:K1:H7 | | O2a, 2b:K5(L):H4 | | O7:K1(L):NM | |
|---|---|---|---|---|---|---|
| | NHS | Peptide #3 | NHS | Peptide #3 | NHS | Peptide #3 |
| CFU | 4.1 | N.D. | 2.9 | N.D. | 5.3 | 2.9 |
| (range) | (4.1-4.2) | | (2.4-3.1) | | (5.2-5.3) | (2.3-3.2) |

N.D. = not detectable

Experiment III

Peptides #1, #3 and #4 were tested for their activity without blood on E. coli O18:K1:H7. The peptides were assayed at 3 μM. The same blood and TFPI controls were included as in Experiment I.

TABLE 4

| | | CFU (range) |
|---|---|---|
| In | Blood control | 5.1 (4.8-5.2) |
| Blood | Peptide #1 | 1.1 (0.4-1.2) |
| | Peptide #3 | N.D. |
| | TFPI | 5.2 |
| | Peptide #4 | 1.3 (1.3-1.4) |
| No | Growth medium | 6.2 |
| Blood | Peptide #1 | 5.6 |
| | Peptide #3 | 6.2 (6.1-6.2) |
| | TFPI | 6.2 |
| | Peptide #4 | N.D. |

N.D. = not detectable

In the absence of blood, the peptides showed little antibacterial activity in E. coli O18:K1:H7. As shown in Table 4, the impact of the peptides on E. coli survival was similar to that of growth medium alone, even at 3000 nM. In contrast, a known antibacterial peptide LL37 (peptide #4; SEQ ID NO: 9) showed full inhibition of bacteria at this concentration. Thus the TFPI-derived peptides may act in cooperation with a factor found in blood to achieve their antibacterial effect.

Experiment IV

The antibacterial effects of peptide #1 (SEQ ID NO: 3), peptide #2 (SEQ ID NO: 8), peptide #3 (SEQ ID NO: 7) and peptide #4 (SEQ ID NO: 9), were further assessed in the following assay.

Time-dependency: Peptides were incubated with 3000 CFU E. coli O18:K1:H7 at the indicated final concentrations in the presence of 10% blood in three parallel 200 μl reactions as described above. RPMI with 10% blood was used as negative control. After incubation for 1, 3, or 5 hours samples were assayed for the effect on bacterial survival by plating serial dilutions onto agar plates. Table 5 demonstrates a time dependency of the bacterial clearance such that no clearance was seen at 1 hour although clearance was demonstrated by peptides #1, #3 and #4 after 3 and 5 hours. The effect of peptides on bacterial survival increases over the time-span studied. Again, peptide #3 showed higher activity than peptide #1, both at 3 and 5 hrs of incubation.

TABLE 5

| | | Peptide #1 | | Peptide #2 | Peptide #3 | | Peptide #4 | |
|---|---|---|---|---|---|---|---|---|
| | Blood | 3 μM | 300 nM | 3 μM | 3 μM | 300 nM | 3 μM | 300 nM |
| CFU 1 hour | 3.4 | 3.4 | 3.4 | 3.4 | 3.2 | 3.3 | 3.1 | 3.3 |
| (range) | (3.2-3.4) | | | | | | | (3.2-3.4) |
| CFU 3 hours | 4.2 | 1.2 | 4.2 | 4.3 | 0.2 | 1.2 | 3.4 | 3.9 |
| (range) | | (1.1-1.3) | (4.1-4.3) | | (N.D.-0.4) | (0.4-1.3) | (3.3-3.5) | (3.4-4.1) |
| CFU 5 hours | 5.1 | 0.2 | 4.2 | 5.2 | N.D. | N.D. | 1.2 | 3.1 |
| (range) | (4.9-5.2) | | (4.2-4.3) | (5.2-5.3) | | | (N.D.-1.4) | (2.9-3.1) |

N.D. = not detectable

The antibacterial effects of peptide #1 (SEQ ID NO: 3), peptide #2 (SEQ ID NO: 8), peptide #3 (SEQ ID NO: 7) and peptide #4 (SEQ ID NO: 9), were further assessed in the following assays.

Capacity: Table 6 demonstrates the capacity of bacterial clearance by bacterial titration. In these experiments, variable concentrations of E. coli O18:K1:H7 (3×10³, 3×10⁴ or 3×10⁵ CFU/200 μl) were challenged with either 3 μM or 100 nM peptide #3 in RPMI/10% blood, and incubated for 3 or 5 hours before dilution and plating. Briefly, the bacteria were diluted to the indicated CFU in 40 μl PBS, the reactions assembled at a final volume of 200 μl as above, and incubated at 37° C. Serial dilutions of the reactions were plated and the colony number determined after overnight incubation. At 3 hours peptide #3 exhibited an effect at all CFU used. At 5 hours no effect on the high CFU culture was discernable, indicating a titration of the killing activity and the high CFU culture exceeding the capacity.

TABLE 6

| | 3000 bacteria | | | 30,000 bacteria | | | 300,000 bacteria | | |
|---|---|---|---|---|---|---|---|---|---|
| | Blood | | | Blood | | | Blood | | |
| | alone | 3 μM | 100 nM | alone | 3 μM | 100 nM | alone | 3 μM | 100 nM |
| CFU 3 hour | 3.9 | N.D. | 2.3 | 5.1 | 1.1 | 1.5 | 7.7 | 5.8 | 7.5 |
| (range) | (3.3-4.2) | | (2.1-2.4) | (4.8-5.1) | (0.4-1.2) | (1.3-1.6) | (7.4-8.1) | (5.7-5.8) | |
| CFU 5 hours | 3.4 | N.D. | 1.2 | 6.3 | 3.6 | 4.1 | 8.4 | 9.1 | 9.1 |
| (range) | (3.2-3.6) | | (N.D.-1.4) | (6.2-6.3) | (2.6-4.1) | (3.6-4.2) | | | |

N.D. = not detectable

### Example 5

### Bacterial Killing Activity is Dependent Upon Active Complement

Experiment I

The TFPI C-terminal peptides have biological activity against Gram negative bacteria (E. coli O17:K1:H7) when in combination with the acellular fraction of blood (plasma or serum) as shown in FIG. 8 A. FIGS. 8A, 8B and 8C depict that the activity can be eliminated by heat treatment of the plasma or serum at 56° C. for 30 minutes and by treatment with cobra venom factor (CVF). CVF has been shown to deplete serum of complement mediated lytic activity by depletion of all terminal complement components [48]. In FIG. 8C, the results are shown for experiments in which serum was treated with either 10 U or 1 U of CVF as follows: A stock solution of CVF at 100 U/ml and 10 U/ml was prepared by dilution in PBS. Serum was treated at room temperature for 30 minutes at a 10:1 ratio with either CVF stock. For control reactions, RPMI was treated with 10 U/ml CVF, and serum was incubated in absence of CVF at room temperature, in parallel. The treated sera were used at 10% of the total reaction volume as before. The reaction with untreated serum was supplemented with 10% CVF-treated RPMI. Controls indicate that peptide #3 is active in untreated serum or in serum that has been incubated for 30 minutes at room temperature without CVF. Activity of peptide #3 was lost in serum that was treated with 10 U/ml of CVF, and thus depleted of complement mediated lytic activity. At 1 U/ml CVF, the killing activity in combination with peptide #3 is still preserved, suggesting that the low concentration is not sufficient to deplete complement activity.

E. coli O18:K1:H7 possess a polysialic acid K1 capsule which is thought to confer resistance to complement mediated killing [49]. The data indicates that cationic peptides derived from the TFPI C-terminus may be able to modify the resistance.

Experiment II

To evaluate if the bacterial killing activity in serum is representative of that in blood, peptides #1, #2, #3, and #5 were serially diluted to 30 M, 300 nM, and 30 nM. As controls serum and serum with 300 nM TFPI were included. As shown in FIG. 8D, peptide #3 in conjunction with serum has the strongest effect, followed by peptide #5, and then by peptide #1 with greatly reduced activity. Peptide #2 is inactive. The peptide activities in serum are at the same magnitude as those previously observed in blood (see Example 4, Experiment I, Table 2).

Experiment III

To evaluate if cathepsin G-digested rTFPI acts in combination with serum components, digested rTFPI at a final concentration of 5 μM was included in bacterial killing assays with blood at 20 μl or serum at 40 μl. Peptide #3 was used in parallel samples. Control lanes indicate blood or serum samples without addition of peptide or digested rTFPI. As is shown in Table 7, protease-digested TFPI acts together with serum components, similar to the TFPI C-terminal peptide.

TABLE 7

| | Blood | | | Serum | | |
|---|---|---|---|---|---|---|
| | Control | Digest | Peptide #3 | Control | Digest | Peptide #3 |
| CFU | 4.1 | 2.2 | 1.5 | 4.2 | 1.2 | 1.2 |
| (range) | | (2.1-2.2) | (1.3-1.8) | (4.1-4.2) | (1.1-1.3) | (0.2-1.4) |

### Example 6

### Identification of Complement Factors Involved in Bacterial Killing Activity

Experiment I

To identify the specific target of synergy with peptide #3, bacterial killing experiments were carried out using serum depleted or deficient for single complement factors. Human sera depleted of C3, C1q-, C2-, C6-, and C9, C4-deficient guinea pig serum (derived from genetically deficient animals) and normal human serum were purchased. Experiments were carried out as above. FIG. 9 A-D depicts the results from experiments wherein serum depleted for complement factors C1q, C3, C6, C2, C9, or deficient for C4 were separately tested. These experiments revealed that removal or lack of the above complement factors resulted in loss of peptide #3 bacterial killing activity. Notably, most depleted sera had some residual killing activity, while the C4-deficient serum was completely devoid of bacterial killing activity, suggesting that incomplete removal of the complement factor may be the cause for residual activity. Furthermore, when purified C1protein complex or factor C4 is added back to the respective reactions at approximately physiological concentrations killing activity is restored (FIGS. 9 C and D). With an assumed serum concentration of 117 μg/ml for C1q, and 310 μg/ml for C4, the reactions were supplemented with 2.34 μg C1complex and 6 μg factor C4, respectively. Factor C1q is the initiator of the classical complement pathway [50]. Factors C6 (C6b, after enzymatic cleavage of C6 into C6a and C6b) and C9 are structural components of the membrane attack complex, which forms the lytic pore responsible for phagocyte-independent killing by complement. Thus, it appears that peptide #3 interacts in some manner with the classical complement pathway, and that the peptide #3 associated complement killing is dependent on formation of the membrane attack complex.

Experiment II

Activation of C1 complex is dependent on Ca²⁺-dependent binding of C1r and C1s, while the lectin and alternative pathway are Ca²⁺-independent, and all complement pathways are Mg²⁺-dependent. To further support the identification of the classical complement pathway as the mediator for the observed bacterial killing activity, a chelation experiment was performed in 10 mM EGTA, supplemented with 5 mM MgCl₂. As shown in Table 8, peptide #3 has no bacterial killing activity in serum containing 10 mM EGTA and 5 mM MgCl₂, while the control reaction in plain serum was active.

TABLE 8

| | Serum | | Serum + 5 mM MgC12, 10 mM EDTA | |
|---|---|---|---|---|
| | Control | Peptide #3 | Control | Peptide #3 |
| CFU | 3.6 | N.D. | 4.4 | 4.2 |
| (range) | (3.4-3.8) | | (4.3-4.5) | (4.1-4.2) |

N.D. = not detectable

### Example 7

### Identification of the Peptide Binding Site

Experiment I

Two heparin binding sites are located at the C-terminus of TFPI and heparin interactions have been noted in clinical studies [51]. Thus, experiments were designed to evaluate the interaction of heparin with the bacterial killing activity. Peptides #1 and #3 were used at 3 μm in experiments as described above, with and without heparin or low molecular weight heparin at 3 U/ml and 0.3 U/ml. Further controls were blood treated under the same conditions in parallel reactions. Un-fractionated heparin at 0.3 U/ml resulted in partial loss of bacterial killing activity of both peptides (data not shown), and 3 U/ml eliminated this activity (see Table 9). The data in Table 9 indicates that the presence of low molecular weight heparin at 0.3 U/mL strongly interferes and at 3 U/mL eliminates the killing activity. This suggests that interactions of the heparin binding site at the C-terminus of TFPI are required for the biological activity of the peptides.

TABLE 9

| | Condition | CFU (Range) |
|---|---|---|
| Blood alone | No heparin | 4.9 |
| control | LMW heparin, 0.3 U/mL | 5.2 (5.2-5.3) |
| | LMW heparin, 3 U/mL | 5.2 (5.2-5.3) |
| | Heparin, 3 U/mL | 5.4 (5.3-5.4) |
| Peptide #1 | No heparin | 1.2 (N.D.-1.4) |
| | LMW heparin, 0.3 U/mL | 4.7 (4.5-5.1) |
| | LMW heparin, 3 U/mL | 5.3 |
| | Heparin, 3 U/mL | 5.5 |
| Peptide #3 | No heparin | N.D. |
| | LMW heparin, 0.3 U/mL | 4.2 (4.1-4.2) |
| | LMW heparin, 3 U/mL | 5.2 (5.2-5.3) |
| | Heparin, 3 U/mL | 5.5 |

N.D. = not detectable

Experiment II

Interactions of the C-terminus of TFPI with LPS have been described [52]. To show direct interaction of peptide #3 with the bacterial cell surface, fluorescent-labeled peptide was incubated with a stock of growing bacteria with and without heparin at increasing concentrations. Details of the experiment are as follows: A frozen stock of E. coli O18:K1:H7 (1×10⁹ CFU/ml) were diluted 1:5 into LB growth medium and incubated for 40 minutes at 37° C. Aliquots of 0.7 ml (3×10^{B} CFU) were washed twice with 10 mM Tris (pH 7.5) and resuspended in 100 μl of 10 mM Tris (pH 7.5) with 10% heat-inactivated serum. Unfractionated heparin was added at a 10-fold concentration to result in 30, 3 and 0.3 U/ml, or was omitted and the samples incubated for 30 minutes at room temperature. Hilyte Fluor™ 555 Dye-tagged peptide #3 was added at a 100 fold (1 μl) concentration to result in 300 nM and incubated in the dark for 5 minutes. The samples were washed twice with 10 mM Tris (pH 7.5), resuspended in 200 μl of 4% paraformaldehyde and incubated for 15 minutes in the dark. After washing in 10 mM Tris (pH 7.5), the samples were resuspended in 100-300 μl and 10 μl loaded onto a cover glass and air-dried. The cover glass was mounted on a slide with mounting media. Microscopy analysis was performed by using a Zeiss Axiovert 200 inverted fluorescent microscope with an AxioCam camera.

As shown in FIG. 10, bacteria incubated with fluorescent labeled peptide #3 without heparin show binding (A). At 0.3 U/ml heparin the fluorescent signal is reduced (B), and at 30 and 3 U/ml heparin eliminates binding (C and D). FIGS. 10 E to 10H show the corresponding Nomarski images.

### REFERENCES (The Contents of Which are Hereby Incorporated by Reference)

[1] EP-0643585

[2] EP-0914830

[3] Piro & Broze, Circulation. 2004 Dec. 7; 110(23):3567-72

[4] U.S. Pat. No. 5,106,833

[5] Wun et al., U.S. Pat. No. 4,966,852

[6] U.S. Pat. No. 5,212,091

[7] U.S. Pat. No. 5,106,833

[8] U.S. Pat. No. 5,888,968

[9] WO 96/40784

[10] Hembrough et al. (2004) Blood 103:3374-80

[11] Bodanszky (1993) Principles of Peptide Synthesis (ISBN: 0387564314).

[12] Fields et al. (1997) Meth Enzymol 289: Solid-Phase Peptide Synthesis. ISBN: 0121821900.

[13] Chan & White (2000) Fmoc Solid Phase Peptide Synthesis. ISBN: 0199637245.

[14] Kullmann (1987) Enzymatic Peptide Synthesis. ISBN: 0849368413.

[15] Ibba (1996) Biotechnol Genet Eng Rev 13:197-216.

[16] Kazmierski (1999) Peptidomimetics Protocols. ISBN: 0896035174

[17] Abell (1999) Advances in Amino Acid Mimetics and Peptidomimetics. ISBN: 0762306149

[18] U.S. Pat. No. 5,331,573

[19] Goodman et al. (2001) Biopolymers 60:229-245

[20] Hruby & Balse (2000) Curr Med Chem 7:945-970

[21] Ribka & Rich (1998) Curr Opin Chem Biol 2:441-452

[22] Kikelj (2003) Pathophysiol Haemost Thromb 33(5-6):487-91

[23] Fareed & Jeske (2004) Best Pract Res Clin Haematol. 17(1):127-38

[24] Chakraborty et al. (2002) Curr Med Chem 9:421-435

[25] Computer-Assisted Lead Finding and Optimization; eds. Testra & Folkers, 1997

[26] Caflish et al. (1993) J. Med. Chem. 36:2142-67

[27] Eisen et al. (1994) Proteins: Str. Funct. Genet. 19:199-221.

[28] Böhm (1992) J. Comp. Aided Molec. Design 6:61-78.

[29] Gehlhaar et al. (1995) J. Med. Chem. 38:466-72.

[30] Moon & Howe (1991) Proteins: Str. Funct. Genet. 11:314-328.

[31] Available from http://chem.leeds.ac.uk/ICAMS/SPROUT.html.

[32] Lauri & Bartlett (1994) Comp. Aided Mol. Design 8:51-66.

[33] Available from Tripos Inc (http://www.tripos.com).

[34] Rotstein et al. (1993) J. Med. Chem. 36:1700.

[35] Lai (1996) J. Chem. Inf. Comput. Sci. 36:1187-1194.

[36] Smith & Waterman (1981) Adv. Appl. Math. 2:482-9.

[37] Needleman & Wunsch (1970) J. Mol. Biol. 48, 443-453.

[38] Rice et al. (2000) Trends Genet 16:276-277.

[39] Gennaro (2000) Remington: The Science and Practice of Pharmacy. 20th edition, ISBN: 0683306472.

[40] Methods In Enzymology (S. Colowick and N. Kaplan, eds., Academic Press, Inc.)

[41] Handbook of Experimental Immunology, Vols. I-IV (D. M. Weir and C. C. Blackwell, eds., 1986, Blackwell Scientific Publications)

[42] Sambrook, et al., Molecular Cloning: A Laboratory Manual (2nd Edition, 1989).

[43] Handbook of Surface and Colloidal Chemistry (Birdi, K. S. ed., CRC Press, 1997)

[44] Short Protocols in Molecular Biology, 4th ed. (Ausubel et al. eds., 1999, John Wiley & Sons)

[45] Molecular Biology Techniques: An Intensive Laboratory Course, (Ream et al., eds., 1998, Academic Press)

[46] PCR (Introduction to Biotechniques Series), 2nd ed. (Newton & Graham eds., 1997, Springer Verlag)

[47] Waage et al. (1989) J. Exp. Med. 169: 333

[48] Gerwurz et al. (1971) Int Arch Allergy Appl Immunol. 1971; 40(1):47-58

[49] Johnson et al. (2001), Journal of Infectious Diseases 183:425-434

[50] Bohana-Kashtan et al. (2004) Molecular Immunology 41: 583-597

[51] Abraham et al. (2003) JAMA 290(2): 238-47

[52] Park et al. (1997), Blood 89: 4268-4274

1. A compound for treating microbial infection in an animal having a complement system, wherein the compound binds to the microbial surface and interacts with components of the complement system present in the animal to kill microbes.

2. The compound as claimed in claim 1 wherein the compound acts synergistically with components of the complement system.

3. The compound as claimed in claim 2 wherein the compound acts synergistically with components of the complement system present in the animal to opsonize microbes.

4. The compound as claimed in claim 2 wherein the compound acts synergistically with components of the complement system present in the animal to cause lysis of microbes.

5. The compound as claimed in claim 1 wherein the microbe is selected from any one of the group consisting of: bacteria, fungi and viruses.

6. The compound as claimed in claim 5 wherein the microbe is Gram negative bacteria.

7. The compound as claimed in claim 1 wherein the compound acts synergistically with the C1 q component of the complement system.

8. The compound as claimed in claim 1 comprising a peptide having an amino acid sequence selected from the group consisting of SEQ ID NOs: 3, 5, 7 and 10, or a peptide having at least 80% identity to any one of SEQ ID NOs: 3, 5, 7 and 10 provided that the polypeptide is not TFPI.

9. A polypeptide comprising an amino acid sequence selected from the group consisting of SEQ ID NOs: 3, 5, 7 and 10, or a peptide having at least 80% identity to any one of SEQ ID NOs: 3, 5, 7 and 10 provided that the polypeptide is not TFPI or a TFPI analog and provided that the amino acid to the N-terminus of SEQ ID NO:3, 5, 7 and 10 is not Lys.

10. The polypeptide as claimed in claim 9, which can bind to LPS and/or to bacteria.

11. The polypeptide as claimed in claim 9, having no more than 50 amino acids.

12. A pharmaceutical composition comprising the compound polypeptide as claimed in claim 9, in admixture with a pharmaceutically acceptable carrier.

13. The pharmaceutical composition as claimed in claim 12 further comprising an antibiotic.

14. The pharmaceutical composition as claimed in claim 12 further comprising TFPI or a TFPI analog, in admixture with a pharmaceutically acceptable carrier.

15. A pharmaceutical composition comprising TFPI or a TFPI analog, and the compound as claimed in claim 1.

16-18. (canceled)

19. A method of screening for bacterial clearance activity comprising the steps of: culturing blood with the polypeptide of claim 9 and a bacterial microbe; and determining the level of bacterial clearance in the blood culture.

20. A TFPI analog, wherein the analog lacks the thrombin cleavage site found near the C terminus of natural TFPI.

21. A TFPI analog, wherein the analog lacks the thrombin cleavage site present between amino acids Lys-254 and Thr-255 of natural TFPI.

22. A TFPI analog, wherein the analog comprises (i) at least one Kunitz domain and (ii) a C-terminal region, but wherein the analog does not have a thrombin cleavage site between its most C-terminal Kunitz domain and the C-terminal region.

23. A TFPI analog, wherein the analog cannot be cleaved by thrombin to give a N-terminal polypeptide that includes a Kunitz domain and a C-terminal polypeptide that does not include a Kunitz domain.

24. A TFPI analog, wherein the analog contains fewer than two Lys-Thr dipeptides. cm 25. A TFPI analog, wherein the analog includes a Kunitz domain 3 of TFPI, but lacks the C-terminus domain of TFPI.

26. A TFPI analog, wherein the analog is a TFPI that has been truncated by up to 23 amino acids from the C terminus.

27. A method of treating microbial infection comprising administering to a subject in need thereof an effective amount of a polypeptide as set forth in claim 9.

28. The method of claim 27, wherein said microbial infection is a bacterial infection.

29. A method of treating a microbial infection comprising administering to a subject in need thereof an affect of amount of a compound as set forth in claim 1.

30. The method of claim 28, wherein said microbial infection is a bacterial infection.
